(19) United States
(12) Patent Application Publication
(10) Pub. No.: US 2024/0420844 A1
(43) Pub. Date: Dec. 19, 2024

(54) SALIVARY SIALIC ACID TESTING FOR CANCER DETECTION

(71) Applicant: Newton Howard, Potomac, MD (US)

(72) Inventor: Newton Howard, Potomac, MD (US)

(21) Appl. No.: 18/745,523

(22) Filed: Jun. 17, 2024

Related U.S. Application Data
(60) Provisional application No. 63/521,645, filed on Jun. 16, 2023.

(51) Int. Cl. G16H 50/20; G01N 21/65; G01N 27/327; G16C 20/20; G16H 50/70

(52) U.S. Cl. CPC G16H 50/20; G01N 21/658; G01N 27/3275; G16C 20/20; G16H 50/70; G01N 2600/00

(57) ABSTRACT

The present systems and methods relate to devices, methods, and systems for detecting the probability of cancer conditions using Sialic Acid concentrations. The present invention relates to a non-invasive system with diagnostic and treatment capacities that use a unified code that is intrinsic to physiological brain function. Embodiments may provide salivary sialic acid testing for cancer detection, for example, breast and oral cancers. Findings indicate the effectiveness of salivary sialic acid testing for detecting solid cancers, potentially serving as an initial screening tool in laboratory settings before invasive diagnostic procedures are recommended.

### CROSS-REFERENCE TO RELATED APPLICATIONS

This application claims the benefit of U.S. Provisional Application No. 63/521,645, filed Jun. 16, 2023, the contents of which are incorporated by reference herein in their entirety.

### BACKGROUND

The present systems and methods relate to devices, methods, and systems for detecting the probability of cancer conditions using Sialic Acid concentrations.

Cancer is a leading public health problem associated with significant morbidity and mortality worldwide. A recent study found that approximately ten million people died from cancer across the globe in 2020, while the number of new cases was around twenty million. Breast cancer was the most prevalent tumor reported, with the disease increasing in recent years. According to the Global Burden of Disease Report, breast cancer accounted for nearly two million cases in 2017. The disability-adjusted life years attributable to breast cancer were estimated to be 17,708,600.

Cancer screening is critical for early diagnosis and access to therapy. A review estimated that the relative reduction in deaths from breast cancer due to early screening is about 20%. The main method of screening for breast cancer is mammography. However, many women experience barriers to breast cancer screening. For example, women who live in remote rural regions are disproportionately impacted by inequitable access to screening. One study found that they were less likely to get a mammogram compared to their non-rural counterparts.

Although early detection via mammography saves lives, regular use of mammographs has its disadvantages, especially exposure to radiation. False positives from mammograms and the need for invasive tissue biopsies are other disadvantages. While the benefits often outweigh the risks, it might not be feasible for some women to perform mammograms regularly. Younger women (i.e., below the age of forty) are also often excluded from screening as for them the risks outweigh the benefits at the population level. However, this practice may lead to missed cases at an individual level. Although rarer, younger women with breast cancer tend to have more aggressive forms of the disease with a higher mortality. Finding less invasive ways to screen them may thus be advantageous. Furthermore, some women experience delays in getting a mammogram, particularly in regions with lower availability.

Patients with other solid tumors, such as oral cancer, may also experience barriers to screening. Dentists are the first point of contact for screening for oral cancer, and not all patients see them for regular checks, including those designated as “high-risk” in one study. Barriers include traveling for remote patients and the costs associated with dental care.

Finding noninvasive ways to pre-screen or adjunct methods of screening that are simple, affordable, and readily accessible is thus important to reduce the barriers to cancer screening. In recent years, the application of biomarkers, such as salivary sialic acid, has increasingly been evaluated for solid tumors. The collection of saliva provides advantages because it is relatively simple and noninvasive and may be used widely in labs. Home collection kits can be sent to patients living in remote areas. There is thus a need to evaluate whether this method is accurate in detecting breast cancer and other solid tumors. Therefore, the rationale for this perspective arises from the imperative to identify noninvasive and easily accessible modalities for cancer screening, specifically those that can serve as pre-screening or supplementary tools alongside existing methods.

### SUMMARY

The present systems and methods relate to devices, methods, and systems for detecting the probability of cancer conditions using Sialic Acid concentrations. The present invention relates to a non-invasive system with diagnostic and treatment capacities that use a unified code that is intrinsic to physiological brain function. Embodiments may provide salivary sialic acid testing for cancer detection, for example, breast and oral cancers. Findings indicate the effectiveness of salivary sialic acid testing for detecting solid cancers, potentially serving as an initial screening tool in laboratory settings before invasive diagnostic procedures are recommended.

For example, in an embodiment, a computer-implemented method for determining a probability of presence of a cancer condition may comprise determining a concentration of sialic acid in a saliva sample of a person using a sialic acid concentration analysis device, comparing the determined concentration of sialic acid with a database relating sialic acid concentrations with cancer conditions, and determining and outputting a probability of presence of a particular cancer condition based on the comparison of the determined concentration of sialic acid with a database relating sialic acid concentrations with cancer conditions.

In embodiments, the sialic acid concentration analysis device may comprise a spectrophotometer. The sialic acid concentration analysis device may comprise a Raman spectrometer. The sialic acid concentration analysis device may comprise an electrochemical sensor.

In an embodiment, a system for determining a probability of presence of a cancer condition may comprise a sialic acid concentration analysis device adapted to determine a concentration of sialic acid in a saliva sample of a person, and a computer system comprising a processor, memory accessible by the processor, and program instruction and data stored in the memory whereby the computer system is adapted to: control the sialic acid concentration analysis device so as to obtain the concentration of sialic acid, compare the determined concentration of sialic acid with a database relating sialic acid concentrations with cancer conditions, and determine and output a probability of presence of a particular cancer condition based on the comparison of the determined concentration of sialic acid with a database relating sialic acid concentrations with cancer conditions.

### BRIEF DESCRIPTION OF THE DRAWINGS

The details of the present invention, both as to its structure and operation, can best be understood by referring to the accompanying drawings, in which like reference numbers and designations refer to like elements.

FIG. 1 is an exemplary illustration of the difference in concentration of sialic acid in cancer patients and healthy control groups.

FIG. 2a is an exemplary illustration of frequency of sialic acid concentrations.

FIG. 2b is an exemplary illustration of frequency of sialic acid concentrations.

FIG. 3 is an exemplary illustration of association between salivary sialic acid levels and oral cancer.

FIG. 4 is an exemplary illustration of a serum levels of TSA and LSA in oral cancer patients.

FIG. 5 is an exemplary illustration of sialic acid concentration (SAC) of groups of patients.

FIG. 6 is an exemplary flow diagram of cancer detection using Sialic Acid concentration.

FIG. 7 is an exemplary block diagram of a computing device, in which processes involved in the embodiments described herein may be implemented.

### DETAILED DESCRIPTION

The present invention relates to a non-invasive system with diagnostic and treatment capacities that use a unified code that is intrinsic to physiological brain function. Embodiments may provide salivary sialic acid testing for cancer detection, for example, breast and oral cancers. Findings indicate the effectiveness of salivary sialic acid testing for detecting solid cancers, potentially serving as an initial screening tool in laboratory settings before invasive diagnostic procedures are recommended.

Salivary sialic acid testing may provide early cancer detection and improved healthcare accessibility. Additionally, portable testing technologies may enhance its role in pre-screening individuals, monitoring treatment response, and facilitating timely referrals for more targeted diagnostic procedures. By addressing barriers to cancer screening, such as invasiveness and limited access to healthcare facilities, salivary sialic acid testing has the potential to revolutionize cancer detection and significantly contribute to saving lives and improving patient outcomes.

There is a strong positive relationship between cancer (oral and breast) and salivary sialic acid levels. This data includes a dose-response relationship, with higher levels of the acid being linked to more advanced stages of cancer.

Salivary sialic acid testing can be a useful method not only to detect cancer and catch early asymptomatic or precancerous stages of the disease but also to monitor the response to treatment. Salivary sialic acid levels declined in patients who had been treated for cancer, so salivary sialic acid may be a good prognostic marker as well as a potential tool to monitor treatment response and disease progression. Furthermore, salivary sialic acid levels may also be useful as pre-screeners for cancer in patients who feel healthy. Following this use, patients may be referred for more diagnostic measures, such as mammograms and tissue biopsies.

By increasing accessibility to timely pre-screening, salivary sialic acid testing could potentially save many lives. Furthermore, mammograms use radiation, which is harmful and can slightly increase the risk of developing cancer in the long run. Thus, women who would otherwise not have undertaken a mammogram (because they belong to a lower-risk group), may find it easier to simply test their saliva for sialic acid levels and then make informed decisions about follow-up imaging and biopsy procedures.

Individuals at risk of oral cancer, including those who chew tobacco or have HPV (which slightly increases the risk of oral tumors), may benefit from simple and noninvasive salivary testing. Dentists are usually the first point of contact for diagnosing oral cancer, but not all patients have equitable access to dental care. By undertaking a simple and noninvasive test, individuals may then gain priority access to oral cancer care, including those who would have avoided visiting the dentist. It is also important to further establish whether both free and bound salivary sialic acid levels should be evaluated, given the potential for false positives in tobacco chewers without cancer. Bound levels may be significantly different in those with cancer versus those without, suggesting that this may be a more accurate measure.

Further, salivary sialic acid testing may be used for detecting ovarian cancer and other solid tumors. After hypothetically using this form of testing, patients can make informed decisions based on their family history, risk factors, and symptoms on whether to undertake further confirmatory and specification tests.

Another important aspect is the threshold for normal levels versus the mean level that indicates an abnormality. Typically, higher levels are found in patients with cancer, especially at later stages. However, it is important to determine diagnostic criteria and variation across the population according to race, ethnicity, and sex. Given the promising results discussed above, it would be beneficial to make this form of testing more available to patients at their medical clinics.

For the assay of free, protein-bound, and/or total salivary SA, techniques such as variations of spectrophotometric assays employing either Gaitonde's acid ninhydrin reagent reaction developed by Yao and colleagues or Skoza and Mohos protocol, which uses periodate, sodium arsenite, and thiobarbituric acid. These methods were developed in 1970-1980s and are well-known for the analysis of SA, and now can be considered reference techniques for detection of this biomarker in saliva.

While quite precise and chemically simple, these methods do have certain disadvantages. Number and complexity of manipulations, special chemical reagents, as well as the need for spectrophotometer to read the final result, make these protocols useable only by trained professionals in a clinical lab setting.

Other detection techniques, such as surface enhanced Raman spectroscopy technique may be used reliably analyze SA in patients. For example, use may be made of surface-enhanced Raman scattering signal of SA absorbed on nanoparticles to generate analytic signal using Raman spectrometer. The method allows much more facile analysis of SA in saliva. Instead of a sequence of chemical manipulations, saliva sample aliquot is just mixed with the nanoparticle solution in specific proportions. This method has great potential to replace quite dated Yao and Skoza+Mohos protocols, offering a low cost and simple assay technique, using a Raman spectrometer.

Example of other techniques that may be used include an electrochemical sensor for highly sensitive SA detection was based on molecularly imprinted polymers, it displayed good selectivity, reproducibility and stability. Such method for SA detection can be portable and adapted for personalized use, as no sample processing is required. A sensor for SA based on optic fibers modified by magnetic nanoparticles was also developed, response time of which amounts to a few seconds, which is orders of magnitude faster than gold standard techniques, which can take up to 40 min per sample. Another state-of-the art device, a capillary sensor based on UiO-66-NH2 metal-organic framework, which has shown operation in micro-volume (15 μl) analysis of SA in a rapid, reliable fashion. All these and many other recent developments in novel SA sensing techniques are yet to find application in clinical validation studies. However, these exciting developments show great promise for translation of salivary SA assay to more portable solutions, which would not require trained professional attention, and ultimately may be used by patients themselves in home settings for potentially real-time analysis of SA levels in saliva for early cancer detection.

Commonalities among the Lowry method, Navazesh method, Nihydrin method, Diphenelyne method, and Yao method include their use of spectrophotometry to measure color changes in the samples. The Lowry method and Navazesh method both measure the amount of protein in the sample by inducing a color change reaction and measuring the resulting color using a spectrophotometer. The Nihydrin method, Diphenelyne method, and Yao method are specifically focused on measuring the amount of sialic acid (SA) in the sample. They all involve the addition of specific chemicals to the sample to induce a color change reaction, which is then measured using a spectrophotometer. In terms of potential for portable or wearable devices, the Yao method may be more suitable. It utilizes Gaitonde's acid ninhydrin reagent and spectrophotometry to measure the color change in the sample. This method has the potential for implementation in portable spectrophotometers, allowing for on-the-go analysis of sialic acid levels. However, it is important to note that the suitability of these methods for portable or wearable devices depends on various factors such as the size, complexity, and power requirements of the spectrophotometric equipment used. Further research and development are needed to optimize these methods for portable or wearable applications, including the exploration of miniaturized spectrophotometry or alternative sensing techniques like electrochemical or optical sensors.

Raman spectrometers have made significant progress towards becoming more portable and handheld, while spectrophotometers require more advanced laboratory infrastructure. Currently, Raman spectrometers have more potential for on-site or in-field analysis. However, both technologies are advancing, and future developments could lead to more portable versions of both instruments.

Both Raman spectrometry and spectrophotometry can provide accurate results for saliva analysis, depending on the specific application and method used. Raman spectrometry offers higher accuracy and specificity for specific molecular analyses, while spectrophotometry is more suitable for overall protein or DNA content measurements.

The surface enhanced Raman spectroscopy technique, as well as developments in molecularly imprinted polymers and optic fibers modified by magnetic nanoparticles, show promise for portable and rapid salivary sialic acid detection. These advancements have potential for future clinical validation studies.

An example of an instrument that may be suitable is shown in U.S. Patent Pub. Nos. 20210285943 A1, 20220178920 A1, 20220187288 A1, 20220214277 A1, 20220178920 A1, 20220187288 A1, 20220214277 A1, all of which are hereby incorporated by reference herein.

Sialic Acid Concentration and Its Correlation with Various Cancers, Cancer Stages, and Classifications

The realm of cancer encompasses a range of diseases characterized by abnormal cell growth, which can infiltrate organs and metastasize to other parts of the body. Cancerous cells have the potential to damage surrounding healthy tissues, resulting in diverse symptoms depending on the specific type of cancer. While some cancers exhibit more readily identifiable symptoms, others present greater challenges for early detection. The mortality rate associated with cancer is alarmingly high, with 18.1 million new cases reported in 2018 and 9.6 million resulting in death. However, certain cancers prove more fatal than others due to various factors, including the effectiveness of disease detection and treatment. For instance, brain cancer demonstrates a meager 5-year survival rate of 12.2%, oral cancer stands at 50%, while testicular cancer boasts an impressive 5-year survival rate of 95.3%.

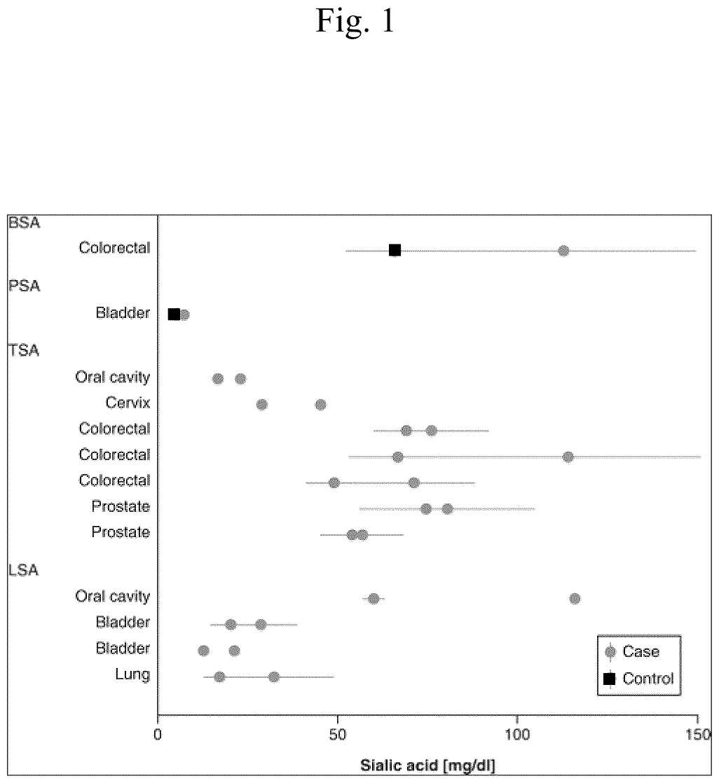

Research encompassing diverse malignancies has suggested that sialic acid may serve as a promising indicator and marker for the presence of these malignancies. Numerous studies across different disciplines have highlighted increased levels of sialic acid in patients compared to healthy individuals, thereby identifying total sialic acid (TSA), bound sialic acid (BSA), and protein-bound sialic acid (PSA) as potential markers for various types of cancer. FIG. 1 is a 2-D plot displaying the difference in concentration of sialic acid in cancer patients and healthy control groups. Moreover, alterations in sialylation levels have been observed in siglecs and other receptors such as EGFR60-62 in lung and colorectal cancers.

TABLE 1
Comparison between sialic acid levels in cancer patients and control groups.

| No. of | Cancer | | | | |
|---|---|---|---|---|---|
| participants | type | Control(mg/dl) | Disease(mg/dl) | p-value | Ref |
| 107 | Lung | LSA: 17.2 (12.7; 21.7) | LSA: 32.4 (16.0; 48.8) | N/A | 47 |
| 118 | Bladder | LSA: 20.3 (14.5; 26.1) | LSA: 28.7 (22.4; 38.9) | 0.001 | 48 |
| | | PSA: 5.6 (4.4;6.7) | PSA: 7.2 (5.9; 8.5) | | |
| 67 | Breast | TSA: 83.6 | TSA: 108 | <0.01 | 49 |
| 60 | Oral | TSA: 29.0 (28.2; 29.8) | TSA: 45.3 (43.8; 46.8) | 0.05 | 50 |
| | | LSA: 16.7 (16.1; 17.4) | LSA: 23.0 (22.1; 23.9) | | |
| 91 | Cervix | TSA: 60.0 (56.9; 63.1) | TSA: 108 | <0.01 | 51 |
| 540 | Prostate | TSA: 54.1 (45.1; 63.1) | TSA: 56.8 (45.3; 68.3) | 0.013 | 52 |
| 111 | Colorectal | TSA: 66.8 (53.1; 80.5) | TSA: 114.1 (77.3; 150.9) | <0.0001 | 53 |
| | | BSA: 65.9 (52.3; 79.5) | BSA: 112.9 (76.1; 149.7) | | |

LSA-lipid-bound sialic acid,
TSA-total sialic acid,
BSA-bound sialic acid,
PSA-protein-bound sialic acid

### Sialic Acid and Breast Cancer

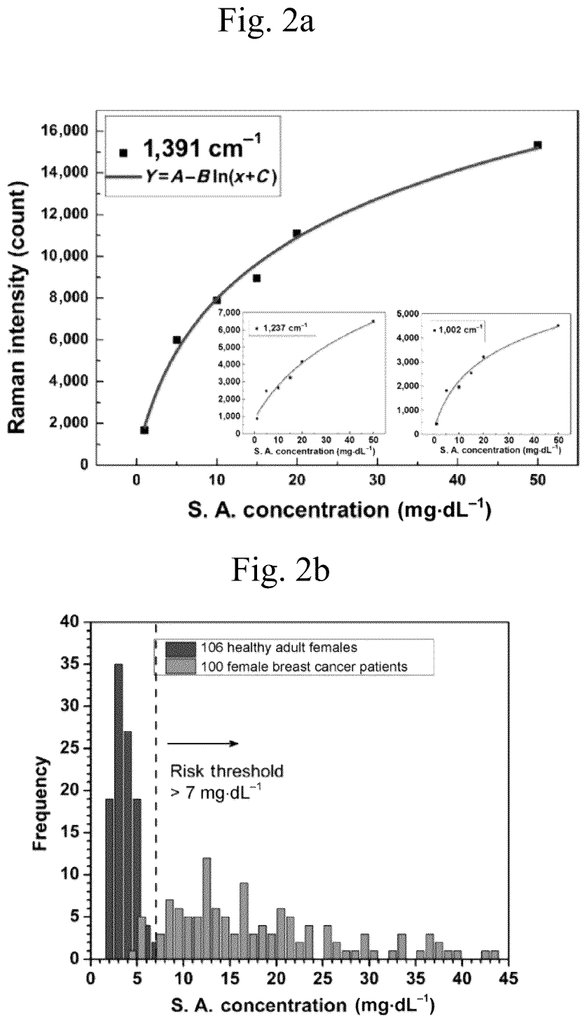

A few studies evaluated the relationship between salivary sialic acid levels and breast cancer. These studies found significantly higher sialic acid levels in patients with breast cancer compared to cancer-free individuals. FIG. 2 shows a plot of frequency of sialic acid concentrations with an interval width of 1 mg·dL⁻¹ for both healthy controls (red bars) and patients with breast cancer, irrespective of cancer stage (green bars). Breast cancer was confirmed in patients using mammography followed by tissue biopsy. The study by Artega et al. found that sialic acid levels were nearly twice as high in patients, with a mean concentration of 14.9 mg/dl versus 6.7 mg/dl (p-value<0.01). This study validated salivary sialic acid, finding a sensitivity of 80% and specificity of 93%. Another study by the authors, which compared levels in 100 breast cancer patients versus 164 healthy individuals, found that the mean salivary sialic acid levels were 18.5 mg/dl in patients versus 3.5 mg/dl in healthy subjects. A study conducted in Turkey by Ozturk et al. found that the mean salivary sialic acid level was 114 mg/dl in patients compared to 47 mg/dl in healthy individuals (p-value<0.01). It should be noted that this study was undertaken in patients with established and pre-diagnosed breast cancer at different stages, which affected the mean level, rather than participants who were screened for cancer during the early stages of the disease.

### Sialic Acid and Oral Cancer

### Staging and Malignancy Indicator

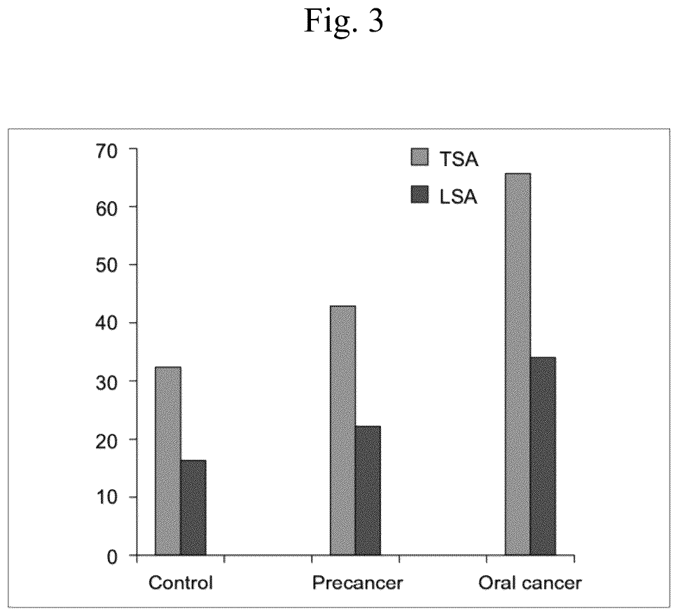

Most of the studies on salivary sialic acid have been undertaken in patients with oral cancer. These studies have all found a significant and positive association between salivary sialic acid levels and oral cancer. FIG. 3 shows the elevation of TSA and LSA levels exhibited statistical significance among untreated oral cancer patients when compared to the control and precancer groups. This suggests that serum glycoconjugates have the potential to serve as a distinct differentiator between oral cancer and precancer conditions.

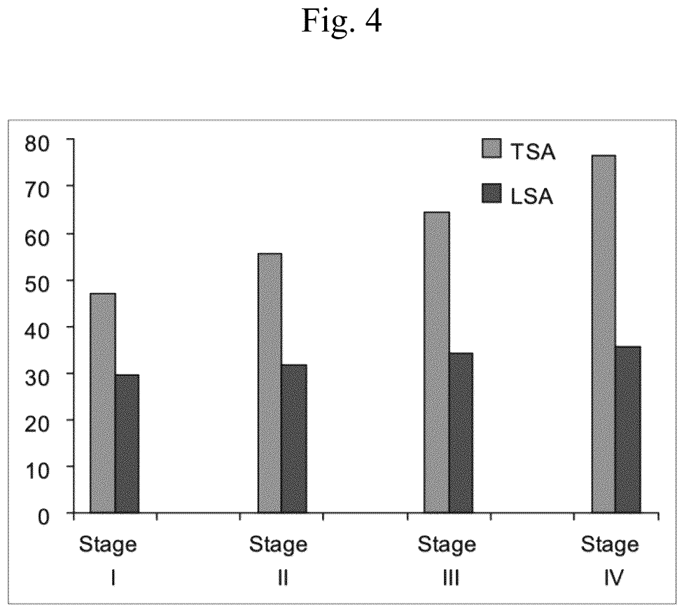

A dose-response relationship was also identified, with higher sialic acid levels linked to more advanced stages of cancer and progression. For example, the study by Hemalatha et al. in India found that salivary sialic acid levels were directly associated with the histopathological grade of the carcinoma, with lower levels in less differentiated tumors compared to more advanced tumor grades. Specifically, patients with well-differentiated oral tumors had mean salivary sialic acid levels of 7.4 mg/dl when compared to patients with less differentiated tumors (6.6 mg/dl) (p-value<0.01); healthy controls had mean levels of 2.1 mg/dl. Similarly, there was a positive relationship between mean salivary sialic acid levels and cancer staging, including marked differences in mean levels in patients with pre-cancerous tumors (57.5 mg/dl) compared to healthy controls (40.3 mg/dl) and patients with established oral cancer (80.4 mg/dl) (p-value<0.01). FIG. 4 shows serum levels of TSA and LSA in oral cancer patients, which demonstrate a progressive increase from stage-I to stage-IV. The highest levels are observed in the most advanced stage of malignancy, indicating a direct correlation with tumor burden, primary lesion stage, and presence of distant metastasis. It should be noted that the study conducted by Poudel et al. in Nepal did not find a significant association between moderately differentiated and late-stage squamous cell carcinomas and salivary sialic acid levels. However, the differences were still significant in patients with squamous cell carcinoma compared to healthy controls.

### Bound Versus Free Salivary Sialic Acid Levels

The study by Hemalatha et al. only found an association with bound salivary sialic acid levels when compared to free levels and oral cancer. However, another study conducted in India, by Sanjay et al., found that both free and bound salivary sialic acid levels were associated with oral cancer, with a dose-response relationship. The study by Azeem et al. found that both tobacco chewers and non-tobacco chewers with oral cancer had increased levels of bound salivary sialic acid, with no significant differences between the two groups. However, oral cancer patients had significantly higher levels of free salivary sialic acid relative to their tobacco-chewing counterparts without cancer (p-value<0.05). Thus, assessing both free and bound levels may be useful in tobacco-using populations (r=−0.5; p-value<0.05). In another study using Kruskal Wallis test significant difference in serum and salivary levels of sialic acid between control, tobacco user, PML, and OSCC groups was demonstrated (P=<0.0001). Table 2 shows significant differences between the four groups in salivary and serum Free Sialic Acid (FSA) and Protein-based Sialic Acid (PBSA).

| | | | | | | | Mann-Whitney U test |
|---|---|---|---|---|---|---|---|
| | | Min | Max | | Chi | | and Bonferroni's |
| Category | N | (mg/dl) | (mg/dl) | Median | Square | P Value | correction test) |
| SalPBSA | Control | 1.00 | 4.00 | 2.10 | 31.34 | <.0001 | Control Vs Other + |
| | Tobacco | 3.70 | 5.90 | 4.40 | | | Tobacco user Vs Oscc |
| | user | | | | | | P < .0001 |
| | PML | 2.10 | 9.40 | 5.20 | | | |
| | OSCC | 4.01 | 7.90 | 5.80 | | | |
| SalPSA | Control | .09 | 3.78 | 1.80 | 24.40 | <.0001 | PML/OSCC Vs Control + |
| | Tobacco | 1.40 | 4.80 | 2.10 | | | PML/OSCC Vs tobacco |
| | user | | | | | | users P < .01 |
| | PML | 2.23 | 4.80 | 3.70 | | | |
| | OSCC | 2.30 | 5.81 | 3.81 | | | |

### Effects of Chemotherapy

The study by Trivedi et al. investigated the effects of chemotherapy on salivary sialic acid levels in patients with oral cancer, finding that patients who had undergone chemotherapy had significantly lower levels compared to cancer patients who had not been treated. Furthermore, Poudel et al. found a significant negative relationship between salivary sialic acid levels and treatment duration.

### Sialic Acid and Other Solid Tumors

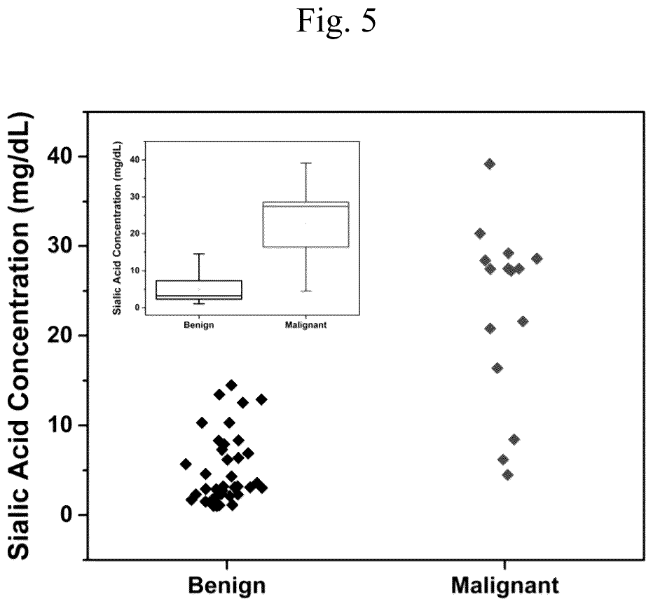

In addition to oral and breast cancers, one study explored the relationship between salivary sialic acid levels and ovarian cancer. The study was conducted by De Jesus et al. in Mexico and found a significant difference in salivary sialic acid levels in benign compared to malignant ovarian cancers. The reported range of sensitivity and specificity was 80-100%, indicating that Sialic Acid levels measured from saliva can be equally good predictor as MRI index for the presence of ovarian cancer in sensitivity while outperforming it in terms of specificity. FIG. 5 shows the sialic acid concentration (SAC) of each group of patients. On the left are the SAC of benign adnexal mass (BAMP) affected patients, and on the right those in which histology has diagnosed ovarian cancer (OCP)

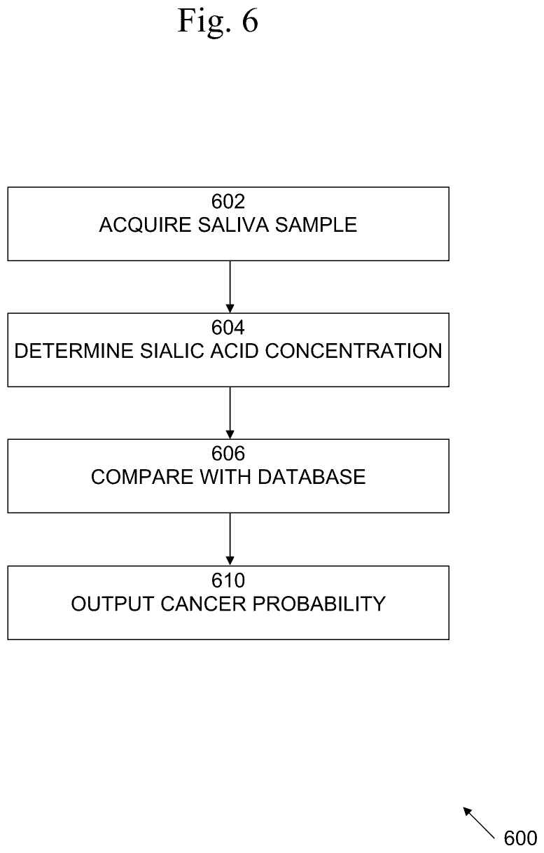

An exemplary process 600 of cancer detection using Sialic Acid concentration is shown in FIG. 6. Process 600 begins with 602, in which a saliva sample may be acquired. At 604, the sialic acid concentration may be determined using one of the techniques described above, or other suitable technique. At 606, the determined sialic acid concentration may be compared with a database relating sialic acid concentrations with cancer conditions. At 610, the probability of presence of particular cancer conditions may be output.

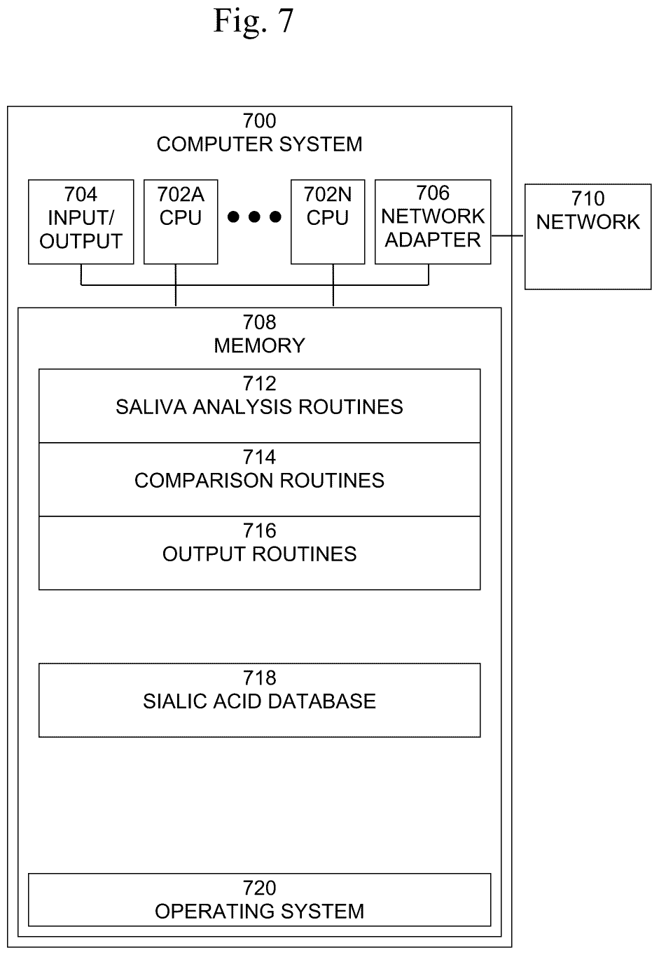

An exemplary block diagram of a computing device 700, in which processes involved in the embodiments described herein may be implemented, is shown in FIG. 7. Computing device 700 may be a programmed general-purpose computer system, such as an embedded processor, microcontroller, system on a chip, microprocessor, smartphone, tablet, or other mobile computing device, personal computer, workstation, server system, and minicomputer or mainframe computer. Computing device 700 may include one or more processors (CPUs) 702A-702N, input/output circuitry 704, network adapter 706, and memory 708. CPUs 702A-702N execute program instructions in order to carry out the functions of the present invention. Typically, CPUs 702A-702N are one or more microprocessors, such as an INTEL PENTIUM® processor, etc. FIG. 7 illustrates an embodiment in which computing device 700 is implemented as a single multi-processor computer system, in which multiple processors 702A-702N share system resources, such as memory 708, input/output circuitry 704, and network adapter 706. However, the present invention also contemplates embodiments in which computing device 700 is implemented as a plurality of networked computer systems, which may be single-processor computer systems, multi-processor computer systems, or a mix thereof.

Input/output circuitry 704 provides the capability to input data to, or output data from, computing device 700. For example, input/output circuitry may include input devices, such as keyboards, mice, touchpads, trackballs, scanners, etc., output devices, such as video adapters, monitors, printers, etc., and input/output devices, such as, modems, etc. Network adapter 706 interfaces device 700 with a network 710. Network 710 may be any public or proprietary LAN or WAN, including, but not limited to the Internet.

Memory 708 stores program instructions that are executed by, and data that are used and processed by, CPU 702 to perform the functions of computing device 700. Memory 708 may include, for example, electronic memory devices, such as random-access memory (RAM), read-only memory (ROM), programmable read-only memory (PROM), electrically erasable programmable read-only memory (EEPROM), flash memory, etc., and electro-mechanical memory, such as magnetic disk drives, tape drives, optical disk drives, etc., which may use an integrated drive electronics (IDE) interface, or a variation or enhancement thereof, such as enhanced IDE (EIDE) or ultra-direct memory access (UDMA), or a small computer system interface (SCSI) based interface, or a variation or enhancement thereof, such as fast-SCSI, wide-SCSI, fast and wide-SCSI, etc., or Serial Advanced Technology Attachment (SATA), or a variation or enhancement thereof, or a fiber channel-arbitrated loop (FC-AL) interface.

The contents of memory 708 may vary depending upon the function that computing device 700 is programmed to perform. One of skill in the art would recognize that these routines, along with the memory contents related to those routines, may typically be included on one system or device, but or may be distributed among a plurality of systems or devices, based on well-known engineering considerations. The present invention contemplates any and all such arrangements.

In the example shown in FIG. 7, memory 708 may include saliva analysis routines 712, comparison routines 714, output routines 716, sialic acid database 718, and operating system 720. For example, saliva analysis routines 712 may include routines that interact with one or more analysis devices to determine the sialic acid concentration in a saliva sample, using one of the techniques described above, or other suitable technique. Comparison routines 714 may include routines to compare the determined sialic acid concentration with a database relating sialic acid concentrations with cancer conditions. Output routines 716 may include routines to generate and output a probability of presence of particular cancer conditions. Sialic acid database 718 may include data relating sialic acid concentrations with cancer conditions. Operating system 720 provides overall system functionality.

As shown in FIG. 7, the present invention contemplates implementation on a system or systems that provide multi-processor, multi-tasking, multi-process, and/or multi-thread computing, as well as implementation on systems that provide only single processor, single thread computing. Multi-processor computing involves performing computing using more than one processor. Multi-tasking computing involves performing computing using more than one operating system task. A task is an operating system concept that refers to the combination of a program being executed and bookkeeping information used by the operating system. Whenever a program is executed, the operating system creates a new task for it. The task is like an envelope for the program in that it identifies the program with a task number and attaches other bookkeeping information to it. Many operating systems, including Linux, UNIX®, OS/2®, and Windows®, are capable of running many tasks at the same time and are called multitasking operating systems. Multi-tasking is the ability of an operating system to execute more than one executable at the same time. Each executable is running in its own address space, meaning that the executables have no way to share any of their memory. This has advantages, because it is impossible for any program to damage the execution of any of the other programs running on the system. However, the programs have no way to exchange any information except through the operating system (or by reading files stored on the file system). Multi-process computing is similar to multi-tasking computing, as the terms task and process are often used interchangeably, although some operating systems make a distinction between the two.

The present invention may be a system, a method, and/or a computer program product at any possible technical detail level of integration. The computer program product may include a computer readable storage medium (or media) having computer readable program instructions thereon for causing a processor to carry out aspects of the present invention. The computer readable storage medium can be a tangible device that can retain and store instructions for use by an instruction execution device.

The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry (such as that shown at 208 of FIG. 2) may include, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims. Further, it is to be noted that, as used in the claims, the term coupled may refer to electrical or optical connection and may include both direct connection between two or more devices and indirect connection of two or more devices through one or more intermediate devices.

What is claimed is:

1. A computer-implemented method for determining a probability of presence of a cancer condition comprising:
determining a concentration of sialic acid in a saliva sample of a person using a sialic acid concentration analysis device;
comparing the determined concentration of sialic acid with a database relating sialic acid concentrations with cancer conditions; and
determining and outputting a probability of presence of a particular cancer condition based on the comparison of the determined concentration of sialic acid with a database relating sialic acid concentrations with cancer conditions.

2. The method of claim 1, wherein the sialic acid concentration analysis device comprises a spectrophotometer.

3. The method of claim 1, wherein the sialic acid concentration analysis device comprises a Raman spectrometer.

4. The method of claim 1, wherein the sialic acid concentration analysis device comprises an electrochemical sensor.

5. A system for determining a probability of presence of a cancer condition comprising:
a sialic acid concentration analysis device adapted to determine a concentration of sialic acid in a saliva sample of a person;
a computer system comprising a processor, memory accessible by the processor, and program instruction and data stored in the memory whereby the computer system is adapted to:
control the sialic acid concentration analysis device so as to obtain the concentration of sialic acid,
compare the determined concentration of sialic acid with a database relating sialic acid concentrations with cancer conditions, and
determine and output a probability of presence of a particular cancer condition based on the comparison of the determined concentration of sialic acid with a database relating sialic acid concentrations with cancer conditions.

6. The method of claim 5, wherein the sialic acid concentration analysis device comprises a spectrophotometer.

7. The method of claim 5, wherein the sialic acid concentration analysis device comprises a Raman spectrometer.

8. The method of claim 5, wherein the sialic acid concentration analysis device comprises an electrochemical sensor.
